(19) United States
(12) Patent Application Publication
(10) Pub. No.: US 2021/0330977 A1
(43) Pub. Date: Oct. 28, 2021

(54) NEUROMODULATION CATHETER

(71) Applicant: Ennovation, LLC, Avon, CT (US)

(72) Inventor: Sanjay K. Sinha, Avon, CT (US)

(21) Appl. No.: 17/367,543

(22) Filed: Jul. 5, 2021

Related U.S. Application Data
(63) Continuation-in-part of application No. PCT/US2020/024435, filed on Mar. 24, 2020.
(60) Provisional application No. 63/048,639, filed on Jul. 6, 2020.
(60) Provisional application No. 62/823,332, filed on Mar. 25, 2019.
(60) Provisional application No. 62/911,001, filed on Oct. 4, 2019.

(51) Int. Cl. A61N 1/36; A61M 25/06; A61M 25/00; A61M 25/01

(52) U.S. Cl. CPC A61N 1/36071; A61N 1/36062; A61N 1/36185; A61M 2025/0037; A61M 25/0026; A61M 25/0105; A61M 25/0606

(57) ABSTRACT

Systems of providing pain control include multi-lumen neuromodulation assemblies with one or more catheters, each having proximal end and a distal end. A single-catheter system with integrated multiple lumens may have a first lumen terminating near the distal end of the catheter and a separate second lumen terminating proximally at a point spaced from the distal end of the first lumen. A catheter-in-catheter design utilizes a second catheter extending through and beyond the distal end of a first catheter to provide a lumen terminating at a point distal from the distal end of the first catheter. The proximal end of at least one lumen includes a connector configured to electrically interconnect an electrode at the distal end of that lumen with a nerve stimulator, such as an electrical pulse generator. At least one of the lumens is also configured to receive liquid medications.

### CROSS-REFERENCE TO RELATED PATENT APPLICATIONS

This patent application is a continuation-in-part of International Patent Application No. PCT/US2020/024435, titled “Neuromodulation Catheter” that was filed on Mar. 24, 2020, and claims priority to U.S. Provisional Patent Application Ser. No. 63/048,639, titled “A Catheter-Through-Catheter Arrangement of Stimulating Catheters for Providing Pain Control by Nerve Block and Neuromodulation of Peripheral Nerves” that was filed on Jul. 6, 2020. International Patent Application No. PCT/US2020/024435 claims priority to U.S. Provisional Patent Application Ser. No. 62/823,332, titled “Neuromodulation Catheter,” that was filed on Mar. 25, 2019 and U.S. Provisional Patent Application Ser. No. 62/911,001, titled “Multi-Lumen, Multi-Electrode Neuromodulation Catheter,” that was filed on Oct. 4, 2019. The disclosures of PCT/US2020/024435, U.S. 63/048,639, U.S. 62/823,332, and U.S. 62/911,001 are incorporated by reference herein in their entireties.

### BACKGROUND

### 1. Field of the Disclosure

Disclosed herein are systems, devices and related methods for providing pain control, more specifically to neuromodulation using an electrically stimulating catheter. Various embodiments of the present disclosure relate generally to systems, devices, and related methods for using the same, for providing pain control and/or relief using neuromodulation of peripheral nerves or the spinal cord. More specifically, particular embodiments of the present disclosure relate to systems, devices, and related methods for operating the same, to treat or provide pain control and additionally by retaining the ability to inject medication for pain control to target neural tissue using single lumen or multi-lumen and single electrode or multi-electrode stimulating catheters. Particular embodiments of the present disclosure relate to systems, devices, and related methods for operating the same, to treat or provide pain control by retaining the ability to inject medication to target neural tissue using two stimulating catheters in a catheter-through-catheter system design. The catheter-through-catheter system can be used for either injecting medication for nerve block or delivering electrical current for neuromodulation or both

### 2. Description of Related Art

Peripheral nerve blocks are powerful methods to control post-operative pain or pain after a surgical procedure. Typically, in such nerve blocks local anesthetic solution is injected around a nerve that innervates a region (surgical site) from which pain is generated. The peripheral nerve blocks effectively block noxious stimuli from being transmitted to the brain, thereby reducing pain sensation.

Pain control may also be achieved by neuromodulation of the nerve by delivering an electrical pulse to a nerve via an electrode or stimulating catheter of varying intensity, frequency and pulse duration to block the gate from transmitting noxious stimuli to pass through the dorsal horn of the spinal cord as postulated in Gate Theory

A peripheral nerve block is performed by inserting a needle through the skin and directed towards the nerve to be blocked. When the needle tip is positioned very close to the nerve, local anesthetic solution is injected to surround (“bathe”) the nerve or plexus of nerves.

The needle is guided in one of two ways toward the nerve. Using ultrasound guidance, the target nerve is visualized with an ultrasound scan. The needle tip is advanced toward the nerve using real-time ultrasound visualization.

Using nerve stimulation, a nerve stimulator, which is an electrical current generator, connects to a block needle on one end and generates a pulse of current. The pulse is usually at 1-5 milliampere, at a frequency of 2-4 Hz, and duration of 0.1-1.0 millisecond. The block needle's shaft is electrically insulated, except for the needle tip, which acts as a unipolar electrode and creates an electric field beyond the needle tip. In this utilization, current travels toward the tip and the surrounding tissue, and returns back to the nerve stimulator through the wire connected to a skin (surface) electrode, thereby completing the circuit. The electric field causes depolarization of the nerve, and innervated muscles twitch. Thus, using a low current, the needle tip is known to be close to the nerve, allowing a clinician to be sure that local solution is injected and will result in a successful nerve block.

In some scenarios, nerve stimulation is combined with live ultrasound guidance to perform nerve blocks.

While nerve blocks successfully mitigate pain, they often last about 24 hours, whereas surgical pain duration may extend for days or weeks following surgery. Nerve blocks are therefore of limited efficacy for extended duration surgical site pain.

In such scenarios, a continuous nerve block may be indicated. A catheter, for example, a thin, flexible, 16-18 gauge catheter, is placed close to a nerve. Typically, a medicinal pump is attached to the catheter, and continuously or intermittently infuses local anesthetic, such as a diluted local anesthetic, to the nerve site. When a medicinal infusion pump is not available, the medicine can be injected as bolus with a syringe through the catheter every few hours to maintain the clinical analgesic effect. In some instances, the infusion pump is attached to the catheter and only gives a pre-set bolus of medication that is triggered by the patient (patient controlled analgesia). This allows for a controlled and continuous dosing of nerve block medication, for 2-4 days post-surgery.

In continuous nerve blocks, it is critical that the catheter tip position is placed very close to the target nerve, in order to infuse local anesthetic solution that can bathe the nerve, thereby maintaining a pain blockade.

Positioning or precisely placing the catheter tip in close proximity to the nerve is often accomplished by using a stimulating catheter. Stimulating catheters are typically a thin and flexible tube, often of 16-18 gauge, or of 18-22 gauge. Such catheters are made of an electrically insulating material, such as polyurethane and silicone or other suitable and/or biocompatible material. A metal conductor may be embedded within the wall of the catheter and exposed at both proximal and distal ends of the catheter.

The proximal end of the stimulating catheter, outside the body, may be connected to a nerve stimulator, such as an electrical pulse generator, via one or more internal, embedded, or external wires connected to one or more metal conductors disposed on the outer wall of the catheter. The distal end serves as a unipolar electrode and is placed near a target nerve. A Touhy needle (a hollow hypodermic needle, slightly curved at one end and suitable for inserting epidural catheters), often 16-18 gauge, is used to place the stimulating catheter close to the target nerve. An example of this placement is shown in FIG. 1, which illustrates the Touhy needle 10 being used to guide the stimulating catheter 12, under the skin 14, and adjacent to the nerve 16.

This placement is accomplished using ultrasound guidance and nerve stimulation. Once the Touhy needle tip 18 is placed in close proximity of the nerve 16 under ultrasound guidance, the stimulating catheter 12, connected to a nerve stimulator 20, such as an electric pulse generator, is passed through the Touhy needle 10. The catheter tip 22 comes out of the Touhy needle 10 close to the nerve 16. A first wire 24 electrically interconnects the nerve stimulator 20 to an electrode 26 affixed to the skin 14. Typically, a conductive gel or other conductive media is inserted between the electrode 26 and the skin 14 to enhance electrical conductivity. A second wire 28 electrically interconnects the nerve stimulator 20 to an electrically conductive portion 30 of the catheter 12. An “electrically conductive portion” may be alternatively referred to as an “electrode”.

Energizing the nerve stimulator 20 creates an electric field in the tissue 32 around the catheter tip 22. When this electric field of appropriate intensity overlaps (or encompasses) the nerve 16 it depolarizes the nerve 16 causing a twitch of the muscles innervated by that nerve 16. A clinician then threads the catheter 12 alongside the nerve 16 while maintaining a muscle twitch. The twitch ensures that the catheter tip 22 is adjacent to the nerve 16 and has not veered from the nerve 16. This method of catheter placement is referred to as a catheter-through-needle technique.

Conventional stimulating catheters, of the type illustrated in FIG. 1, have one terminal opening 34 and one electrode 30 at the distal tip 22. If liquid medication exits via that terminal opening 34, the ongoing neuromodulation is usually interrupted either because of dispersion or dilution of the electricity (e.g., electric field) in the liquid medication or because the liquid medication (e.g., a local anesthetic solution) blocks the Sodium-Potassium pump (e.g., receptor on nerve cell or neuron), thereby preventing further transmission of the nerve impulse. Neuromodulation would resume only after the effects of the injected medication have resolved.

Some specific catheter-through-needle type catheters have been previously described. U.S. Pat. No. 7,386,350 to B. Vilims describes such a catheter, or lead, having a single “central lumen that communicates with various infusion ports spaced at selected locations along the lead”. The catheter, or stimulation lead, may also have “a plurality of circumferentially extending electrodes . . . spaced longitudinally along the distal portion”. In such a design it is “preferable to utilize a single conductor along the major length of the lead and then provide branch conductors . . . that then extend to contact the various electrodes”. Such a design, which poises each longitudinally-spaced electrode at the same applied polarity, is “especially adapted for treatment of intervertebral disc ailments”.

In some instances, it would be desirable to provide a stimulating catheter having multiple lumens that terminate at varying distances from multiple electrodes so that neuromodulation may be maintained and uninterrupted by using at least one of the electrodes for neuromodulation and a lumen for injection of a medication (e.g., local anesthetic solution) that is away from the “active” electrode. It would be further desirable to utilize the stimulating catheter for maintaining uninterrupted delivery of neuromodulation to a patient while simultaneously being able to inject an adjuvant or medication to supplement the neuromodulation without reducing or negatively impacting the neuromodulation's effectiveness.

In some instances, it would be desirable to use two stimulating catheters inserted in a catheter-through-catheter arrangement whose lumens do no communicate with each other and their distal tips (openings and corresponding electrode) terminate at varying distances from each other on the target nerve. The two stimulating catheters can be placed from a single needle using one needle puncture site in which one catheter is placed using catheter-through-needle technique (as described above) and the second catheter is inserted using a catheter-over-catheter technique. Once the needle is withdrawn leaving the two catheters in place in a catheter-through-catheter arrangement, it is possible to inject medication from the opening of one catheter while continuing to neuromodulate with an electrode on the second catheter that is farther away (at a distance) from the opening on the first catheter or vice-versa.

It would be further desirable to utilize the stimulating catheters for maintaining uninterrupted delivery of neuromodulation to a patient with one catheter while simultaneously being able to inject an adjuvant or medication to supplement the neuromodulation through the second catheter without reducing or negatively impacting the neuromodulation's effectiveness. In certain clinical situations, one or both of the stimulating catheters can be used initially for injecting local anesthetics medication for surgical anesthesia or analgesia; when the effects of the medication have worn off, neuromodulation can then be initiated from one or both of the stimulating catheters. In addition, one or both of the catheters can be used initially for neuromodulation and if neuromodulation does not produce adequate analgesia, medication (e.g., local anesthetic) can be injected to supplement the neuromodulation as a bolus through one or both catheters.

### BRIEF SUMMARY

Embodiments of the present disclosure relate to, among other things, systems, devices, and related methods for operating the same, to treat or provide pain control by neuromodulation and by retaining the ability to inject medication to target neural tissue using either a single lumen or a multi-lumen and single electrode or multi-electrode stimulating catheter (e.g., multi-lumen, multi-electrode neuromodulation catheter). Embodiments of the present disclosure describe multi-electrode catheters designed to include either 1-3 electrodes, of which one or two or all may be used to provide neuromodulation. The third electrode is not associated with an opening and is used only for neuromodulation or to serve as an electrode to complete the electrical circuit for delivering a neuromodulation.

Embodiments of the present disclosure also relate to, among other things, systems, devices, and related methods for operating the same, to treat or provide pain control by injecting medication for nerve block and neuromodulation and retaining the ability to inject medication to target neural tissue using two stimulating catheters in a catheter-through catheter arrangement. Embodiments of the present disclosure describe a first catheter that has one electrode close to the opening at the distal tip of the catheter and a second catheter that has two electrodes with one electrode at the distal tip of the catheter close to the opening and another electrode on the catheter shaft positioned about 3 cm proximal from the tip. This second electrode on the shaft of the catheter is not associated with an opening and may be used purely for neuromodulation or serve as an electrode to complete the circuit.

Some embodiments of the present disclosure relate to a catheter. The catheter may be a single lumen, single electrode or a multi-lumen, multi-electrode neuromodulation catheter. This neuromodulation catheter may be used for neuromodulation only, for injection(s) of medication only, or for a combination thereof.

Some embodiments of the present disclosure relate to two stimulating catheters a catheter-through-catheter arrangement that maybe used for neuromodulation only, for injection(s) of medication only, or for a combination thereof.

Each of the embodiments disclosed herein may include one or more of the features described in connection with any of the other disclosed embodiments.

It may be understood that both the foregoing general description and the following detailed description are exemplary and explanatory only and are not restrictive of the invention, as claimed.

### BRIEF DESCRIPTION OF THE DRAWINGS

FIG. 1 schematically illustrates placement of an electrically stimulating catheter as known from the prior art.

FIG. 2 illustrates in partial cross-section a single lumen catheter in accordance with a first embodiment.

FIG. 3 illustrates a single lumen catheter in accordance with a second embodiment.

FIG. 4 illustrates an embodiment where a catheter is aligned by passing over a needle.

FIG. 5 illustrates an embodiment where the catheter includes a plurality of lumens and electrodes (multi-lumen, multi-electrode stimulating catheter).

FIG. 6 illustrates a multi-lumen, multi-electrode stimulating catheter to provide neuromodulation.

FIGS. 7A-7C are cross-sectional views of the catheter of FIG. 6 showing different wire and lumen configurations.

FIG. 8 illustrates a stimulating catheter that may be used as an outer catheter in a catheter-through-catheter embodiment, the stimulating catheter having an injection port on or near its hub or proximal end connecting to the lumen that opens at each end of the catheter and an electrode near the tip or distal end.

FIG. 9 illustrates a half-needle, or a needle that has been split in half along its long axis including its hub, which may be inserted into an outer catheter in a catheter-through-catheter embodiment.

FIG. 10 illustrates a second stimulating catheter having a continuous lumen from its proximal and distal ends that may be used as an inner catheter in a catheter-through-catheter embodiment, the catheter having two electrodes disposed near the tip or distal end and a slidable connector engageable with the hub of a half-needle or outer catheter.

FIG. 11 illustrates an assembly of an outer or first stimulating catheter, a half-needle such as the half-needle shown in FIG. 9, and an inner or second stimulating catheter prior to or during insertion into a patient. The hub of the half-needle seats within the hub of the outer catheter and extends beyond the end of the outer catheter. The second or inner catheter is inserted inside of the outer catheter and half-needle in a first position where its distal end or tip extends beyond the distal end or tip of the outer catheter but not beyond the tip of the needle.

FIG. 12 illustrates an assembly of an outer or first stimulating catheter and an inner or second stimulating catheter after insertion into a patient. The half-needle, which was present during insertion, has been removed from the assembly. The second or inner catheter is inserted inside of the outer catheter in a second position where its distal end or tip extends further beyond the distal end or tip of the outer catheter and may be positioned in any appropriate position for innervation and/or neuromodulation.

The accompanying drawings, which are incorporated in and constitute a part of this specification, illustrate exemplary embodiments of the present disclosure and together with the description, serve to explain the principles of the disclosure.

### DETAILED DESCRIPTION

While the present disclosure is described herein with reference to illustrative embodiments for particular applications, it should be understood that embodiments of the present disclosure are not limited thereto. Other embodiments are possible, and modifications can be made to the described embodiments within the spirit and scope of the teachings herein, as they may be applied to the above-noted field of the present disclosure or to any additional fields in which such embodiments would be of significant utility.

In the detailed description herein, references to “one embodiment,” “an embodiment,” “an example embodiment,” etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described.

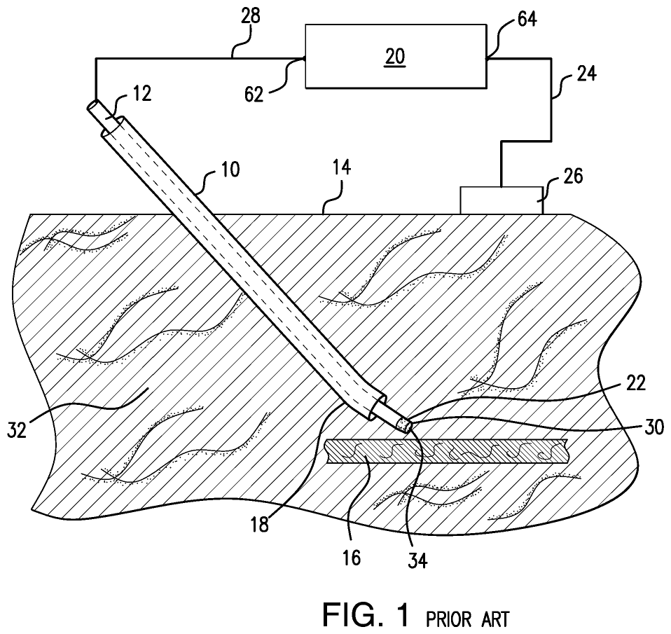

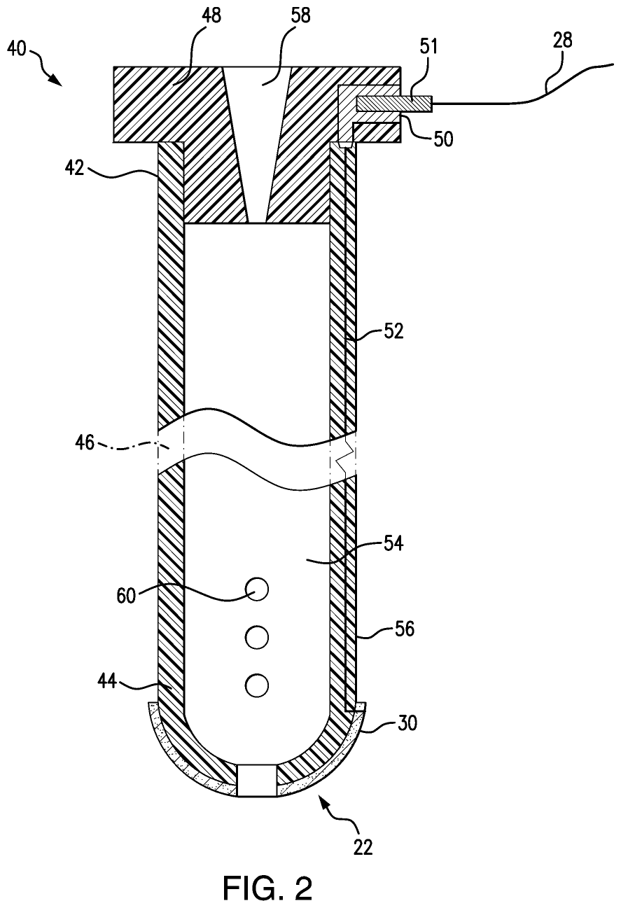

Disclosed herein is a catheter 40 for neuromodulation that is effective to control or reduce pain (FIG. 2). Features discussed herein are located at the proximal end 42 and at the distal end 44 of the catheter. An extended intermediate portion 46, that may be of any required length, is shown in broken phantom lines only. The neuromodulating catheter 40 includes a connector 48. The connector 48 connects at the proximal end 42. When the catheter is inserted to a position similar to that shown in FIG. 1, the connector 48 is positioned outside the body, above the skin 14. The connector 48 may include a contact post 50 to electrically connect with wire 28 extending from the nerve stimulator 20. Wire 52 extends from contact post 50 to the electrically conductive portion 30 of the catheter tip 22. The connector 48 thereby electrically interconnects the electrically conductive portion 30 of the catheter 40 to the nerve stimulator 20 to enable delivery of electrical signal and thereby cause neuromodulation. Wire 52 may extend within the catheter 40 lumen 54. Alternatively, the wire 52 may be embedded in a wall 56 of the catheter. As a further alternative, the wire 52 may be an electrically conductive layer coating a portion of an interior surface of the wall 56.

In an embodiment, the connector 48 further includes an injection port 58. The port 58 enables injection of medication or other fluids. This may be used as an adjuvant to improve analgesic efficacy. As discussed, the connector 48 may be used to connect to currently available stimulating catheters 12, allowing for them to be repurposed for neuromodulation. Exemplary catheters commercially available are Stimucath (manufactured by Arrow), ContiplexStim (manufactured by BBraun), Sonolong, and E-Cath, (manufactured by Pajunk). Medication may be injected through the neuromodulation catheter 40. This provides a benefit of using medications to supplement neuromodulation and increase analgesic efficacy by combining two modalities (medication and neuromodulation) for pain control. Some known methods of neuromodulation utilize solid leads, without a lumen, which does not include a port for injecting medication. While other leads may have lumens, they are not designed for injecting medications, and are not suitable for doing so. Therefore, this embodiment provides for a device and method with a port 58, which allows for medication injection via a lumen 54 and utilizes neuromodulation to increase analgesic efficacy.

The catheter 40 may include a plurality of holes 60 formed through the wall 56 and at the catheter tip 22, and/or along the body of the catheter, adjacent the catheter tip, for example, from 2 millimeters to 2 centimeters away from the catheter tip 22. The multi-holed catheter is specifically formed to deliver medications, and the position of the holes 60 may be in any desired configuration. The catheter tip may be metalized 30 and effective to function as a unipolar lead. Medication or liquid injected from injection port 58 will exit at the holes 60 and at the opening at the catheter tip 22

Utilizing the catheter 40 with the system of FIG. 1, the nerve stimulator 20 is electrically interconnected to the contact post 50. The nerve stimulator 20 may connect to the connector 48 either via jack 51, or directly without use of a jack. An effective nerve stimulator or electrical pulse generator 20 is capable of providing an electrical pulse of 0.1-30 mAmps, with a frequency of 2-10,000 Hz and pulse duration of 0.1-1000 microseconds. The nerve stimulator 20 may be attached to the skin 14 via a paste, glue or medical adhesive. Alternatively, the pulse generator 20 may be located at a different location, such as a pocket, tabletop or clip, and connected via wires 24, 28 to the catheter 40 and/or connector 48. The nerve stimulator 20 may be programmed or instructed via a user interface, on the device itself, or via a remote computer or hand-held controller.

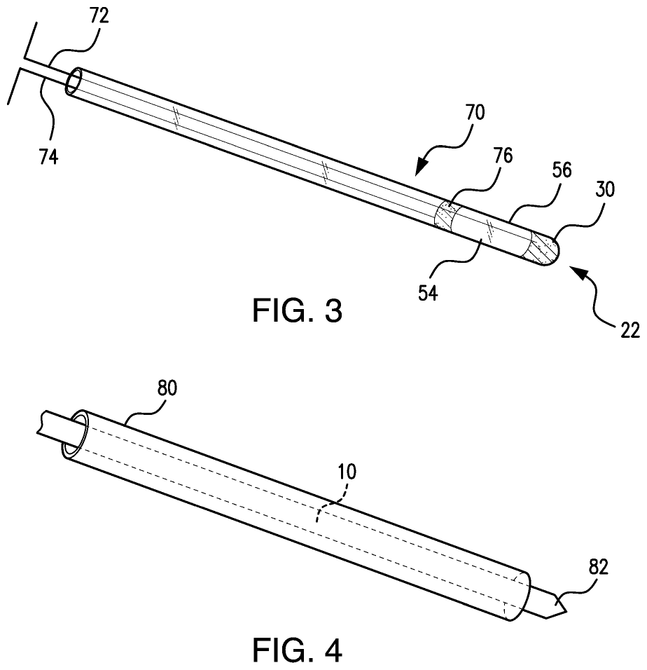

In the above embodiment, one terminal 62 (FIG. 1) of the nerve stimulator 20 is electrically interconnected to the electrically conductive portion 30 of the catheter 40 (FIG. 2). A second terminal 64 (FIG. 1) is electrically interconnected to the skin 14 external to the subject's body completing an electrical circuit. Alternatively, as illustrated in FIG. 3, the catheter 70 includes two wires 72, 74 each independently either extending down lumen 54, embedded in wall 56 or coated on an interior surface of wall 56. One wire 72 terminates at electrically conductive portion 30 at the catheter tip 22. The other wire 74 terminates at a second electrically conductive portion 76 formed on the wall 56 of the catheter 70 at a location spaced from the catheter tip 22. The electrically conductive portions 30 and 76 of the catheter 70 function as circumferential electrodes. While the spacing between electrically conductive portion 30 and second electrically conductive portion 76 may be any effective distance, an exemplary distance is between 0.5 centimeters and 2 centimeters. These wires are attached to the anode terminal and cathode terminal of the nerve stimulator to provide electrodes of different polarity. In this multi-electrode system, a return electrode on the skin is not be required to complete the circuit. This removes the need for a grounding electrode on the surface of the skin.

As described above, the catheter 40, 70 is positioned by being threaded through a pre-positioned needle. Alternatively, as illustrated in FIG. 4, a catheter 80 may be formed to go over the needle 10, as opposed to through the needle. The catheter 80 may be placed close to a nerve with the catheter-over-needle technique. Utilizing the catheter over the needle, enables preloading of the catheter 80 over the needle 10 with the needle tip 82 remaining exposed. The catheter 80 is then connected to a nerve stimulator and advanced into place toward a nerve under ultrasound guidance. As the catheter 80—needle 10 unit is advanced, an electrical pulse is transmitted to the needle tip 82 from a nerve stimulator. This results in a muscle twitch. Once the needle tip 82 is properly in place adjacent to the nerve, the catheter 80 is slid over the needle 10 and advanced alongside the nerve. Subsequently, the electrodes 76 and 30 can be connected to a nerve stimulator, as described above to elicit a muscle twitch providing additional confirmation that the catheter is positioned close to the nerve.

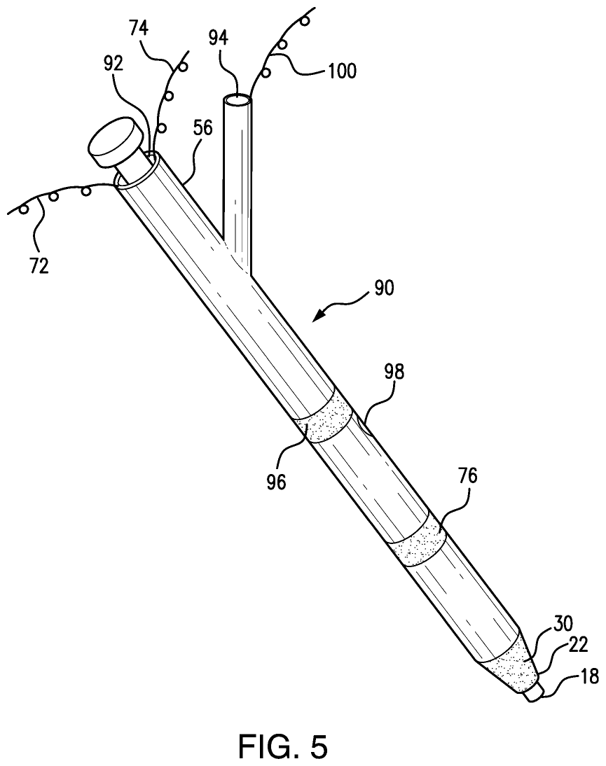

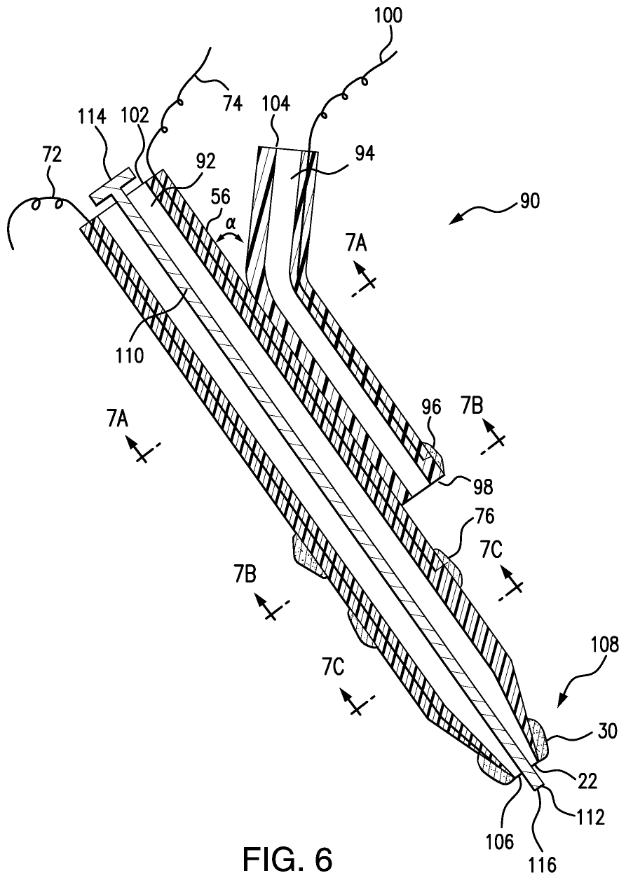

Referring now to FIGS. 5 and 6, the catheter 90 may include a plurality of lumens. Illustrated are a central lumen 92 and peripheral lumen 94 formed adjacent wall 56 of the catheter. Multiple lumens provide multiple channels for delivering current, medications and suitable forms of energy, such as light or sound, to modulate nerves. The central lumen 92 terminates at the catheter tip 22 and provides access for needle tip. A first electrically conductive portion 30 is adjacent the catheter tip 22 and electrically connected a nerve stimulator via wire 72. A second electrically conductive portion 76 is spaced from the first electrically conductive portion and electrically connected to a nerve stimulator, other power source or ground via wire 74. A third electrically conductive portion 96 is located adjacent a distal opening 98 of peripheral lumen 94. This third electrically conductive portion 96 is electrically interconnected to a nerve stimulator, other power source or ground via wire 100.

Shown in FIG. 6 is a cross-sectional view of the catheter 90 with multiple lumen 92,94. Multiple electrodes 30,76,96 are located along the length of the catheter 90 and/or clustered toward the distal tip 22. This provides an ability to neuromodulate a larger area of tissue. The multiple electrodes 30,76,96 are connected to conducting wires 72,74,100 either extending through the lumens 92,94 embedded in wall 56 or coated on an inside surface of wall 56. Interconnecting the wires 72,74,100 will cause the multiple electrodes 30,76,96 to be of the same polarity. Additionally, each of the electrodes 30, 76, 96 can be connected to the electrical pulse generator via wires 72,74,100 in a manner that they are of different polarities.

The multi-lumen simulating catheter 90 has a distal end 22 and a proximal end 102. Once the multi-lumen stimulating catheter 90 has been placed in a proper location near a nerve, it is electrically interconnected to an electrical pulse generator (nerve stimulator 20 of FIG. 1) via wire 72. The catheter 90 has at least two lumens, such as central lumen 92 and peripheral lumen 94. Lumens 92, 94 are preferably not in fluid communication with each other. Each lumen may include a connector attached or secured to its proximal end 102,104. Each lumen 92, 94 may further include an injection port at the proximal end 102, 104 for injecting a medication or liquid (e.g., a local anesthetic solution, opioids, steroids, alpha-2 agonists, Naᵥ 1.7 antagonist or any other suitable liquid medicament for curbing pain in a patient). Medication or liquid injected at proximal end 102 of first lumen 92 will exit out of catheter tip 106 while medication injected at proximal end of second lumen 94 will exit out of tip of second lumen 98. Each lumen may also be configured and dimensioned such that a nerve stimulator (e.g., electrical pulse generator) may be electrically interconnected to the proximal end 102,104 via wires 72,74,100 and electrically interconnected to an electrode 30,76,96.

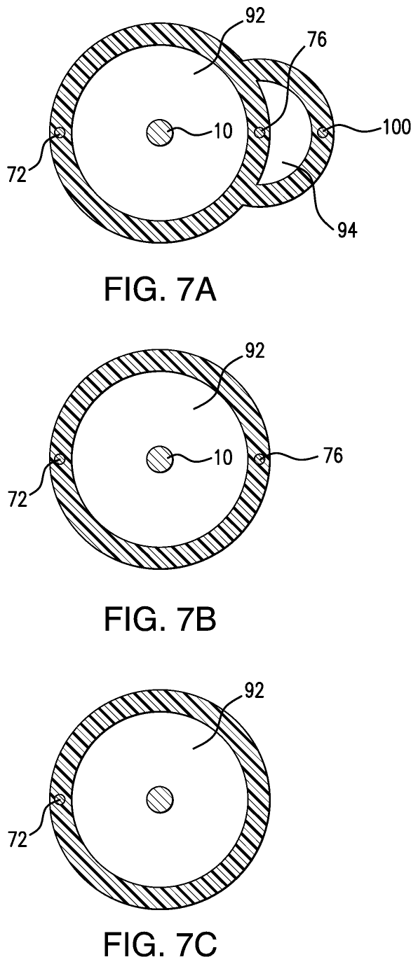

FIGS. 7A-7C are cross-sectional views of the catheter 90. FIG. 7A shows a central lumen 92 and peripheral lumen 94 along with wires 72,74,100. FIG. 7B shows central lumen 92, first wire 72 and second wire 74. FIG. 7C shows central lumen 92 and first wire 72.

Referring back to FIG. 6, the central lumen 92 extends from proximal end 102 to an opening 106 at the distal end 108. A liquid or medicament may be injected at the proximal end 102 of this central lumen 92 and will exit at the opening 106. The peripheral lumen 94 may terminate at different or variable distance from the distal end 108.

The peripheral lumen 94 proximal end 104 may be disposed at an angle, α, relative a longitudinal axis of the central lumen 92. The peripheral lumen 94 may be configured and dimensioned to terminate at a distal opening 98 that is approximately 3 cm proximally away from the distal opening 106 of the central lumen 92. In some embodiments, this second distal opening 98 is spaced from 1 cm to 6 cm proximally away from the distal opening 106. In an exemplary use case of this catheter 90, when a liquid or medicament is injected through this peripheral lumen 94, the liquid or medicament may exit through the opening 98 3 cm proximally away from the distal tip 106 of the multi-lumen catheter 90. The multiple lumens 92,94 may be separated by a partition 56. The partition 56 may be integral with the construction of the multi-lumen catheter such that the partition and the catheter walls are formed as a single piece. In some embodiments, the partition may be a distinct and independent structural divider to prevent fluid communication between the multiple lumens.

A needle 110 may be disposed through the central lumen 92. The needle 110 extends from an opening at the proximal end 102 and extends through the entire length of the central lumen 92 and exits through the opening 106 at the distal end 108. The needle 110 may be used to place or position the multi-lumen catheter 90 inside a subject (e.g., inside a patient's body). The needle 110 may be electrically insulated along a portion of the shaft with only a tip being an exposed electrical conductor. In such a scenario, only the needle tip 112 is electrically conductive, while the reminder of the needle 110 is insulated and not conductive. The needle tip 112 is shown in FIG. 6 extending beyond the distal end 108 of the central lumen 92. In this embodiment, the needle tip 112 is electrically conductive. The nerve stimulator may be attached or secured to the proximal end 114 of the needle 110. The tip 112 may be blunt so that injury to target nerves is minimized (e.g., a Touhy needle or a blunt bevel needle). In an exemplary embodiment, for “catheter-over-needle” insertion techniques, the needle 110 includes a lumen 116 for injecting liquid medications.

It can be appreciated that in other embodiments, additional lumens may be disposed or used, each including an opening that is respectively proximally spaced away from the distal tip of the catheter with varying distances (e.g., the third lumen 4 cm from the distal tip, the fourth lumen 1 cm from the distal tip, etc.). Each of these lumens may include their own separate injection port.

During operation of the multi-lumen catheter, a current may be passed through the catheter, thereby causing neuromodulation of the adjacent nerves. In an exemplary embodiment of the multi-lumen catheter, a wire may be used to connect (e.g., electrically connect) the nerve stimulator (e.g., electrical pulse generator), pass axially along the length of one of the lumens, and connect the stimulator to electrodes disposed at the distal ends of the catheter lumen. In an embodiment, the nerve stimulator (e.g., electrical pulse generator) may include an electrode in the neuromodulating catheter and then connect to a skin electrode of opposite polarity to complete the circuit.

In another embodiment, the second conductive portion 76 functions as a reference electrode, or electrode of opposite polarity to complete a circuit. The peripheral lumen 94 may be used to deliver liquid medication. The central lumen 92 and the peripheral lumen 94 are not in fluid communication. Because the lumens 92, 94 are not in fluid communication, liquid medicament exiting at distal opening 98 will not cause dispersion of electricity thereby not interrupting the ongoing neuromodulation. As previously described, ongoing neuromodulation may be interrupted either because an injection of a liquid or medicament may cause dispersion of the electricity stemming from the nerve stimulator (e.g., the electric field delivery becomes weakened) or because the liquid or medicament may block the Na—K receptors and prevent further transmission of the nerve impulse. It is advantageous to use the multi-lumen stimulating catheter described throughout the present disclosure to overcome these challenges and to provide continuous, uninterrupted neuromodulation. Furthermore, the multi-lumen stimulating catheter, as taught herein, may be configured and dimensioned to connect to the nerve stimulator, taught herein, to cause a tiny current to pass through the catheter, thereby neuromodulating the nerves, without interruption of the neuromodulation therapy.

In an exemplary embodiment, the multi-lumen catheter 90 may include additional electrodes disposed in close proximity to each of the distal end openings 106, 98 of the respective lumens. For example, the electrode 30 is positioned at the distal end 106 of the central lumen 92 and electrically interconnected to the wire 72. The secondary electrode 76 may be separately positioned near the distal end and electrically interconnected to the second wire 74. In an exemplary embodiment, the second electrode 76 that does not have a corresponding lumen, opening or injection port and functions as a reference electrode or electrode of opposite polarity to complete the circuit. Each of the wires 72, 74, 100 may be connected electrically to the nerve stimulator. In some embodiments, the electrodes 30, 76, 78 may be connected by separate wires 72, 74, 100 embedded in a wall 56 of the multi-lumen catheter 90 to corresponding connectors at the proximal ends of the multi-lumen catheter. The wires may be secured in or to the wall. In an exemplary embodiment, each wire may be embedded in the wall of the catheter and be electrically insulated.

In addition, there may be a weave pattern of metal wires (braiding) to maintain stiffness of the catheter and patency of the catheter and lumen. However, this weave pattern may not be responsible to maintain electrical conductivity between the nerve stimulator and the electrodes.

In an embodiment, each of these electrodes 30, 76, 78 may be separately connected to a nerve stimulator at corresponding proximal ends of the catheter 90 via a connector and may function as independent electrodes. In other words, the central lumen electrode 30 may function as a first independent electrode and the peripheral lumen electrode 78 may function as a second independent electrode, not coupled or restricted to the first electrode 30.

In an exemplary embodiment, a connecter may be attached or secured to a proximal end of the multi-lumen stimulating catheter. Each of the plural lumens may have its own connector. The connectors may be configured to permit each of the respective proximal ends of the plural lumens to be connected to the nerve stimulator. Each connector also includes one or more delivery ports or injections ports to allow for injections of a liquid or medication.

A nerve stimulator or an electrical pulse generator is used to provide the neuromodulating when operating the multi-lumen stimulating catheter. In an exemplary embodiment, the electrical pulse generator may deliver a current of varying intensity ranging from approximately 0.01 to 30 Milliamperes, a frequency of approximately 1 to 4999 Hertz, and a pulse duration of approximately 1 to 1000 microseconds to the electrodes of the catheter. For placement of the catheter, and to elicit a muscle twitch, the pulse generator may be set at approximately 1 to 2 Hertz, an intensity of approximately 1 to 5 Milliamperes, and a pulse duration of approximately 0.05 to 1 millisecond.

Once the catheter is placed, neuromodulation may be initiated. The intensity of the current may be gradually increased until a twitching or a buzzing (paresthesia) sensation is felt in the appropriate dermatome or the region of the body that is being targeted. This current intensity may have a range of approximately 0.01 to 30 Milliamperes. When the twitching of a muscle group is required, the frequency of the electrical pulse may be set between approximately 1 to 6 Hertz. If a buzzing sensation is required, the frequency may be set between approximately 10 to 4999 Hertz.

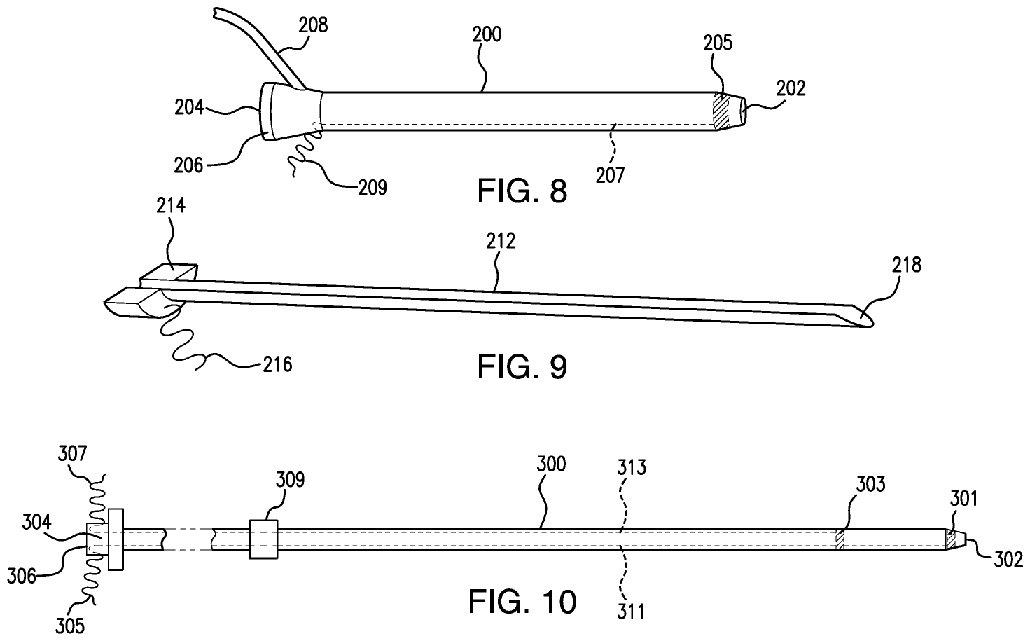

In an exemplary catheter-through-catheter embodiment, as shown in FIG. 8, a first or outer stimulating catheter 200 has a lumen with a distal opening 202 and a proximal opening 204 with a hub 206. There is an injection port 208 near the hub 206 that connects with the lumen of the catheter and allows injection of a medication solution into the catheter. There is an electrode 205 at the distal end or tip of the catheter 200 connected to an outer lead or connector 209 by an embedded conductor or wire in the catheter wall, external, or internal wire 207. The outer lead or connector 209 may electrically connect with an electrical pulse generator. In some embodiments, the length of the catheter 200 may vary in length from about 3 cm to about 25 cm, or from about 5 cm to about 15 cm.

FIG. 9 illustrates a half-needle 212, or a needle that has been split in half along its long axis including its hub 214, which may be inserted into an outer catheter in a catheter-through-catheter embodiment. The proximal end having a hub 214 may be electrically attached to a lead or connector 216 which may in turn electrically connects with an electrical pulse generator. The shaft of the needle is electrically insulated with only the distal tip 218 exposed such that when an electrical pulse generator is connected to electrical connector 216 the current flows through the shaft of the needle and exits at the exposed distal tip.

A second or inner stimulating catheter 300, illustrated in FIG. 10, has a lumen with a distal opening 302 at the distal end of the catheter and a proximal opening, or port 306, in a hub 304 at the proximal end of the catheter. The catheter may be constructed with various lengths, as denoted by the phantom lines in FIG. 10. A liquid medication solution may be injected into and through the catheter lumen via the port 306. The catheter has two electrodes 301 and 303 with one of the electrodes 301 adjacent to the opening 302 at the tip or distal end of the catheter. In an embodiment, the other electrode 303 is positions approximately 3 cm proximal from the distal catheter tip. In some embodiments, the electrode 303 is about 0.5 cm, or about 1 cm, or about 1.5 cm, or about 2 cm, or about 2.5 cm, or about 3 cm, or about 3.5 cm, or about 4 cm, or about 4.5 cm, or about 5 cm, or about 5.5 cm, or about 6 cm, or about 7 cm, or about 8 cm, or about 9 cm, or about 10 cm proximal from the distal catheter tip. The electrodes are connected by separate wires, 311 and 313, that are embedded in the wall of the catheter and exit at the hub 304 near the proximal end of the catheter. The wires 311 and 313 are electrically connected to leads and/or connectors 305 and 307, which can in turn be connected independently with one or more electrical pulse generator(s). A slidable connector 309 slides over the catheter 300 and is designed to fit inside the hub 206 of the first or outer catheter 200 when catheters 200 and 300 are assembled.

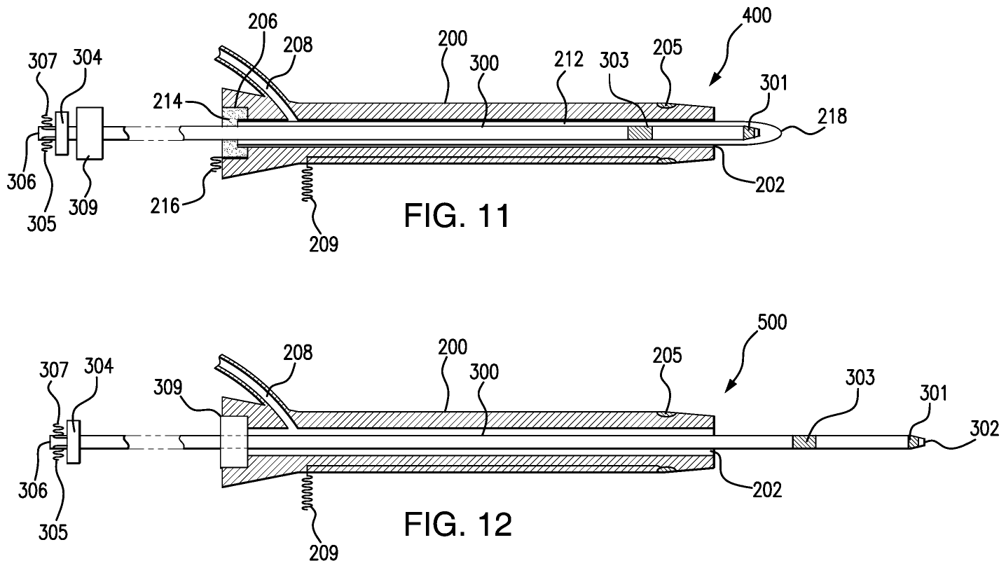

An assembly 400 of the first or outer catheter 200, the half-needle 212 and the second or inner catheter 300, is illustrated in FIG. 11. The half-needle 212 is inserted through the proximal end of the outer catheter 200 such that its hub 214 is retained within the hub 206 of the outer catheter and such that the tip 218 of the half-needle 212 extends through the lumen of the catheter 200, and through and beyond the opening 202 at the distal end of the catheter 200. The needle may be designed at different lengths such that its tip protrudes from the distal end of the catheter 200 to varying degrees. In an embodiment, the needle tip protrudes about 0.25 cm beyond the distal end of the catheter. In some embodiments, the needle tip protrudes about 0.05 cm, or about 0.1 cm, or about 0.15 cm, or about 0.2 cm, or about 0.25 cm, or about 0.3 cm, or about 0.35 cm, or about 0.4 cm, or about 0.45 cm, or about 0.5 cm, or about 0.55 cm, or about 0.6 cm, or about 0.7 cm, or about 0.8 cm, or about 0.9 cm, or about 1 cm, or about 2 cm, or about 3 cm beyond the distal end of the catheter.

The second or inner catheter 300 may be inserted into the assembly 400 with the needle 212 in place within the outer catheter 200. The slidable connector 309 on the inner catheter 300 allows the second catheter to be placed in a range of positions relative to the outer catheter 200 and needle 212. In a first position, as illustrated in FIG. 11, the distal end of the inner catheter extends through and beyond the distal opening 202 of the outer catheter but does not extend beyond the tip of the half-needle 218. The inner catheter may be retained in the first position by positioning the slidable connector 309 to rest against the hub 214 of the half-needle inserted in the outer catheter 200. The inner catheter may be in the first position during insertion and advancement of the needle towards a nerve.

An assembly 500 of the outer catheter 200 and inner catheter 300 excludes the half-needle 212, that is, the half-needle 212 has been removed after insertion of advancement of the needle towards a nerve. The slidable connector of the inner catheter 309 is designed to fit inside the hub 206 of the outer catheter. With the needle 212 removed, the hub 206 of the outer catheter may accept the slidable connector 309 of the inner catheter 300 in order to prevent the catheters 300 and 200 from sliding/moving in relation to each other, once they have been placed in their final position along the nerve. The slidable connector 309 may be disengaged from the hub 206 of the outer catheter 200 in order to make adjustments in the position of the inner catheter 300 as necessary.

In an exemplary embodiment, a pump (not depicted) may be provided for injecting an infusion or bolus of medication through the injection port(s). The pump may be incorporated into the design of the nerve stimulator so that the combined device may deliver the electrical impulses for neuromodulation and an injection of medication. In some embodiments, the pump may be programmable. For example, the pump may be programmed to deliver a continuous infusion of medication or a bolus of medication or a combination of both. A computer, one or more processors, and one or more interfacing units may be used to control the pump's programming.

With the foregoing designs and embodiments described herein, it can be appreciated that in using a multi-lumen, multi-electrode stimulating catheter, it becomes possible for a user (e.g., physician or other healthcare provider) to inject a small aliquot of liquid or medication (e.g., local anesthetic solution, opioids, steroids, alpha-2 agonist, etc.) through one opening (e.g., proximal or distal port/opening) of the first lumen, while continuing neuromodulation using an electrode that is far away from the opening through which the liquid or medication is exiting. To maintain uninterrupted neuromodulation, the electrode that is positioned closer and/or nearer to the spinal cord of a patient or the central nervous system will be attached to the nerve stimulator (e.g., electrical pulse generator), as taught herein. Concurrently, an injection of a liquid or medicament may be administered from the opening that is distal, or farther away, from the spinal code or central nervous system. This is an appreciable advantage of using the multi-lumen stimulating catheter, as taught herein, over traditional stimulating catheters.

Methods or techniques of placing and/or positioning the multi-lumen stimulating catheter close to a neural tissue (e.g., spinal cord, central nervous system, etc.) are also now described. It can be appreciated that other methods or techniques of placing the multi-lumen stimulating catheter, as taught herein, may be used, and the described methods herein are in no way limiting.

### Catheter-Over-Needle

An exemplary first use case for positioning the multi-lumen stimulating catheter is now described. With a catheter-over-needle technique, and at a first step, the multi-lumen catheter may be pre-loaded over an insulated needle such that the tip of the needle is exposed beyond the length of the catheter. At a next step, the needle may be attached to a nerve stimulator (e.g., electrical pulse generator). As the catheter-needle unit is advanced under ultrasound guidance towards a nerve, and the needle tip is in close proximity to the nerve, the electrical pulse transmitted to the needle tip to the target nerve will elicit an appropriate muscle twitch indicating close proximity of the needle tip to the nerve. After which, at a subsequent step, the nerve stimulator (e.g., electrical pulse generator) may be disconnected from the needle and connected to the distal electrode of the catheter tip. At a step, the catheter may be slid over the needle and advanced alongside the nerve. As the catheter slides alongside the nerve, an appropriate muscle twitch will be maintained thus confirming that the catheter tip is near or in close proximity to the nerve. At a subsequent step, the nerve stimulator (e.g., electrical pulse generator) may then be disconnected from the distal tip electrode and attached to the proximal electrode. If the proximal electrode is close to the nerve, then an appropriate muscle twitch will again be elicited. If there is no muscle twitch, the catheter can then be advanced further until a twitch is elicited. In an exemplary embodiment, the needle is an insulated needle whose tip is bare and capable of conducting electricity.

### Catheter-Through-Needle

An exemplary second use case for positioning the multi-lumen, multi-electrode stimulating catheter is now described below step-by-step. With a catheter-through-needle technique, and at a first step, an insulated needle, with a tip that is bare and capable of acting as a unipolar electrode, may be attached to a nerve stimulator (e.g., electrical pulse generator), and the needle tip may be positioned in close proximity to the target nerve under ultrasound guidance. When the needle tip is in close proximity to the nerve, an appropriate muscle twitch may be elicited. At a subsequent step, the nerve stimulator (e.g., electrical pulse generator) may be disconnected from the needle and attached to the catheter at its proximal end to electrify the distal electrode (e.g., at the catheter tip), and the catheter may then be threaded through the needle. As the tip of the catheter exits from, or comes out of, the needle tip, an appropriate muscle twitch may be elicited. At a step, the catheter may be threaded alongside the nerve while maintaining the twitch. At a step, once the catheter has been advanced 3 cm beyond the needle tip, the nerve stimulator (e.g., electrical pulse generator) may be disconnected from the distal electrode and attached to the proximal electrode. If the proximal electrode is in close proximity to the nerve, then a muscle twitch may also be elicited. If there is no twitch, the catheter should be advanced further until a muscle twitch is elicited. Subsequently, the needle is withdrawn leaving the catheter in place.

### Exemplary Use Case of the Multi-Lumen Catheter

The following steps may be used to use the multi-lumen, multi-electrode catheter. At a first step, once the catheter is placed properly using one of the placement techniques described herein, the electrode that is closest to the spinal cord or the central nervous system may be attached to a nerve stimulator (e.g., electrical pulse generator) and stimulated with a current of varying intensity ranging from approximately 0.1 to 30 milliamps, at a frequency ranging from approximately 2 to 4999 Hertz, and a pulse duration ranging from approximately 0.1 to 1000 microseconds. To complete the circuit, either a surface skin electrode is used or two electrodes of different polarity (e.g. anode and cathode) are used to complete the circuit. In an exemplary embodiment, a third electrode may be used that is not associated with a lumen and may act as the second electrode to complete the circuit.

At a second, optional, step, if pain control is not adequate, a small aliquot of medication (e.g., a local anesthetic solution, opioids, steroids, alpha-2 agonists, Na_{V} 1.7 antagonist or any other suitable liquid medicament for curbing pain in a patient) may be slowly injected through the injection port taught herein such that medication will exit from the hole that is farther away from the electrode that is being used for neuromodulation. In doing so, the injected medication will not disperse the electricity delivered during the neuromodulation and the medication will not block the Na—K pump (receptor) or another receptor depending on the medication injected and modulate or prevent further transmission of the nerve impulse and thereby reduce pain. In other words, the neuromodulation will be maintained and uninterrupted by using the multi-lumen stimulating catheter as described above.

It will be appreciated that the multi-lumen stimulating catheter described herein provides a multitude of benefits over traditional stimulating catheters and their techniques for using the same. An advantage of the multi-lumen stimulating catheter described herein is that it allows for neuromodulation of peripheral nerves and spinal cord to occur uninterrupted while retaining the ability to inject medication through the catheter. Another advantage is that the multi-lumen stimulating catheter may be placed with the catheter-over-needle assembly or through a pre-positioned the needle. Yet another advantage is that the multi-lumen stimulating catheter may include two or more electrodes that may be used for neuromodulation of peripheral nerves or cranial nerves (e.g., vagus, trigeminal, hypoglossal, etc.) or nerves at or surrounding the spinal cord. These electrodes may deliver electrical stimulation (neuromodulation) from one or all of the electrodes simultaneously or deliver the electrical stimulus from only one electrode while the other one is silent or of different polarity.

Another advantage of the multi-lumen stimulating catheter described herein is that it includes two or more separate injection ports for injecting medication that connect separately to their corresponding openings on or at the distal end of the catheter that are positioned close to the electrodes. Each of these openings includes a separate lumen that may connect to the proximal injection ports. These lumens do not communicate with each other. Yet another advantage of the multi-lumen stimulating catheter system described herein is it may be used for electrically stimulating neural tissue using one electrode while simultaneously retaining the ability to inject medication through an opening that is close to the electrode that is not electrically stimulated (i.e., the silent or not active electrode).

Yet another advantage of the multi-lumen stimulating catheter system described herein is that the electrodes, as taught herein, may be stimulated from approximately 1 to 4999 Hertz (Hz) to neuromodulate the nerves.

Yet another advantage of the multi-lumen stimulating catheter system described herein is that the catheter, as taught herein, may be fabricated or designed with multiple electrodes and openings. In an exemplary embodiment, the distance between the electrodes and/or the openings may vary from approximately 0.5 cm to 10 cm.

Another advantage of the multi-lumen stimulating catheter system described herein is that the opening(s) and the electrode(s), as taught herein, may be positioned in various configurations such that each electrode may be closely associated with an opening, or along the catheter shaft, in different patterns (e.g., a hole on alternate electrodes or holes between two electrodes, etc.).

An advantage of the multi-lumen stimulating catheter described herein is that it provides a system that combines a nerve stimulator (e.g., electrical pulse generator) and an infusion pump into a single, combined device that may connect to a proximal end of a catheter and allow neuromodulation and injection of medication to occur simultaneously or independent of each other.

### Use of the Catheter-Through-Catheter System

To prepare the assembly 400 for insertion, the needle 212 is inserted inside the outer catheter 200. The tip of the needle 218 protrudes from the opening 202 of the catheter to expose the sharp tip of the needle beyond the catheter opening 202. The hub of the needle 214 engages with and fits in the hub 206 of the outer catheter 200

The catheter 300 is then inserted inside the half-needle 212 and placed in a first position such that the tip of the catheter is inside the semi-circular space defined by the half-needle and is not beyond the tip of the half-needle 218. In some embodiments, to prevent movement or sliding of the inner catheter 300 relative to the outer catheter 200 and needle 212, the stopper/slidable connector 309 may be placed against, locked against, or engaged with the hub 214 of the needle. In some embodiments, to prevent movement or sliding of the inner catheter 300 relative to the outer catheter 200 and needle 212, the stopper/slidable connector 309 may be configured to lock/fit inside the hub 214. In some embodiments, to prevent movement or sliding of the inner catheter 300 relative to the outer catheter 200 and needle 212, the connector 309 may be configured to engage with the remaining half of the space within the hub 206 of the outer catheter not occupied by the half-hub 214 of the needle. The stopper/slidable connector 309 may be configured and dimensioned in any appropriate manner to prevent movement or sliding of the inner catheter 300 relative to the outer catheter 200 and needle 212 when engaged with the outer catheter 200 and/or needle 212.

With the assembly 400 prepared, the port 306 in the hub 304 is available for injecting fluid through the inner catheter 300 and out of the distal opening 302. The injection port 208 of the outer catheter 200 is available for injecting fluid into and through the lumen of the outer catheter 200 completely separate from the lumen of the inner catheter 300. The fluid injected into and through the outer catheter 200 would exit the opening 202 at the distal end of the outer catheter, localizing the fluid in a more proximal location than any fluid injected into the inner catheter 300.

When the assembly 400 is prepared, i.e. when the outer catheter 200 is positioned over the needle 212 and the inner catheter 300 is positioned through the needle 212, the unit is ready for insertion.

Prior to insertion of the assembly 400, the needle 212 is connected to an electrical pulse generator via electrical connection 216. To complete the electrical circuit, a skin electrode is connected to the electrical pulse generator. The current will flow from the electrical pulse generator via the connector 216 to the tip of the needle and into the tissues, and return to the electrical pulse generator via the skin electrode. The needle 212 is insulated except for at its tip 218 such that the current does not exit the needle prior to reaching the tip.

The assembly 400 is inserted into the patient and advanced to a target nerve under ultrasound guidance. As the needle tip is positioned in close proximity to the nerve, an appropriate muscle twitch will be elicited. The electrical pulse generator is then disconnected from the needle 212 and connected to the electrical connection 209 of the outer catheter 200 to electrify the electrode 205 and obtain muscle twitch as was recorded from the stimulation from the needle tip.

The electrical pulse generator is then connected to the catheter 300 via electrical connector 305 which connects to the electrode 301. The stopper 309 on the catheter 300 is unlocked from the hub 214 of the needle to allow free sliding movement of the catheter 300 inside the needle 212 and outer catheter 200.

The catheter 300 is then slid forward from the first position through the needle tip 218 to a second position alongside the nerve. As the catheter sides alongside the nerve, twitching of the muscle is maintained confirming that the catheter tip 302 is next to the target nerve. The second position may be any position in-between the first position and the mechanical limit of the inner catheter (i.e. when the hub of the inner catheter cannot move further past the slidable connector 309). In some embodiments, the tip of the catheter 302 may be pushed about 1-12 cm, or about 1-10 cm, or about 2-8 cm, or about 3-8 cm beyond the needle tip 218. In some embodiments, the tip of the catheter 302 may be pushed about 3-8 cm beyond the needle tip 218. In some embodiments, the tip of the catheter 302 may be pushed about or at least about 1 cm, or about 2 cm, or about 3 cm, or about 4 cm, or about 5 cm, or about 6 cm, or about 7 cm, or about 8 cm, or about 9 cm, or about 10 cm beyond the needle tip 218.

Subsequently, with the inner catheter 300 in a second position, the needle 212 is slid out of the assembly 400 leaving the catheter 200 in place and the catheter 300 inside the catheter 10. Once the needle is removed, the slidable connector 309 may be positioned such that it is engaged with the hub 206 of the outer catheter 200, locking the position of catheter 300 (in a second position) relative to catheter 200. This assembly 500 is shown illustrated in FIG. 12.

With the catheter through catheter deployed, the electrodes 205, 303 and 301 are positioned in different locations along the nerve.

It should now be possible to independently stimulate the nerves with electrodes 205, 303 and 301 and elicit a muscle twitch.

It should also be possible inject medication through catheter 200 from injection port 208 that comes out of opening 202, and separately through injection port 306 on hub 304 which comes out of opening 302.

This arrangement of the electrodes placed in different location on the nerve and the position of the openings on the two catheters make it possible to separate the neuromodulation electrodes from the opening in the catheter from which medication exits out.

This array/arrangement allows for injection of medication from opening 202 while neuromodulation can be maintained from electrodes 303 and 301.

Similarly, neuromodulation can be maintained from electrode 205 and 303 while the medication is injected from the opening 302.

To maintain uninterrupted neuromodulation, separation of the electrodes from the opening where medication comes out is important.

It is also important to use and electrode that is closest to the spinal for neuromodulation and use the opening for injection of medication that is farther away from the spinal cord (and the neuromodulating electrode).

The following steps may be implemented in using the catheter-through-catheter, assemblies as taught herein. At a first step, once the catheter is placed properly using the placement technique described herein (i.e. assembly 500 has been placed), the electrode that is closest to the spinal cord or the central nervous system may be attached to a nerve stimulator (e.g., electrical pulse generator) and stimulated with a current of varying intensity ranging from approximately 0.1 to 30 milliamps, at a frequency ranging from approximately 2 to 4999 Hertz, and a pulse duration ranging from approximately 0.1 to 1000 microseconds. To be able to complete the circuit, either a surface skin electrode is used as described during catheter placement or two electrodes are used, the two electrodes being of different polarity (e.g., anode or cathode), to complete the circuit. In an exemplary embodiment, the proximal electrode 303 on the inner catheter 300 that is not associated with an opening may act as the second electrode to complete the circuit.

At a second and optional step, if pain control is not adequate, a small aliquot of medication (e.g., a local anesthetic solution, opioids, steroids, alpha-2 agonists, or any other suitable liquid medicament for curbing pain in a patient) may be slowly injected through the injection port of the catheter taught herein such that medication will exit from the hole that is farther away from the electrode that is being used for neuromodulation. In doing so, the injected medication will not disperse the electricity delivered during the neuromodulation and the medication will not block the Na—K pump (receptor) and prevent further transmission of the nerve impulse. In other words, the neuromodulation will be maintained and uninterrupted by using the catheter-through-catheter system as described above.

It will be appreciated that the catheter-through-catheter system described herein provides a multitude of benefits over traditional single stimulating catheters and their techniques for using the same. An advantage of the catheter-through-catheter system described herein is that allows for neuromodulation of peripheral nerves and spinal cord to occur uninterrupted with one of the catheters while retaining the ability to inject medication through the second catheter. Another advantage is that the catheter-through-catheter system may include one or multiple electrodes from both the catheters may be used for neuromodulation of peripheral nerves or cranial nerves (e.g., vagus, trigeminal, hypoglossal, etc.) or nerves at or surrounding the spinal cord (spinal nerves or dorsal root ganglion). These electrodes may deliver electrical stimulation (neuromodulation) from one or multiple electrodes simultaneously or can deliver the electrical stimulus from only one electrode while the other electrodes are silent or of different (opposite) polarity.

Another advantage of the catheter-through-catheter system described herein is that it includes two separate injection ports from each catheter for injecting medication that connect to their corresponding openings at the distal end of each of the catheters that are positioned close to their corresponding electrodes. These lumens do not communicate with each other.

Yet another advantage of the catheter-through-catheter system described herein is that one of the catheters may be used for electrically stimulating neural tissue using one electrode while simultaneously retaining the ability to inject medication through an opening that is on the second catheter whose electrode is not being electrically stimulated (i.e., the silent or not active electrode). The design enables separation by variable distance the site of neuromodulation and the site of medication delivered on the nerve.

Yet another advantage of the catheter-through-catheter system described herein is that the electrodes, as taught herein, may be stimulated from approximately 1 to 4999 Hertz (Hz) to neuromodulate the nerves.

Yet another advantage of the catheter-through stimulating catheter system described herein is that the catheter, as taught herein, may be fabricated or designed with multiple electrodes and openings. In an exemplary embodiment, the distance between the electrodes and/or the openings may vary from approximately 0.5 cm to 10 cm.

Another advantage of the catheter-through-catheter system described herein is that the opening(s) and the electrode(s), as taught herein, may be positioned in at variable distances (from each other) along the nerve. Also the configurations of the electrodes and the openings maybe such that each electrode may be closely associated with an opening, or along the catheter shaft, in different patterns (e.g., a hole on alternate electrodes or holes between two electrodes, etc.).

An advantage of the catheter-through-catheter system described herein is that is provides a system that combines a nerve stimulator (e.g., electrical pulse generator) and an infusion pump into a single, combined device that may connect to a proximal end of a catheter and allow neuromodulation and injection of medication to occur simultaneously or independent of each other.

Several alternative embodiments for the catheter-in-catheter system are contemplated. In an alternative embodiment, the outer catheter 200 may have none, one or more than one electrode, with additional electrodes being positioned more proximal to the hub 206 compared to the most distal electrode, with each electrode being independently connected to a wire/connector such that each can be unused or placed at a certain polarity. In an alternative embodiment, the outer catheter 200 may have one or more holes in-between the proximal and distal ends such that liquid medication can be dispensed along portions of the length of catheter 200. In an alternative embodiment, the inner catheter 300 does not have a proximal hub and instead terminates with the same dimensions as the catheter 300. It can be appreciated that the full scope of use of such alternative embodiments would be evident to a person of skill in the art based upon the disclosure herein.

In another embodiment, the inner catheter does not have a lumen or associated opening at its distal end and is used only for delivering electrical stimulation for neuromodulation only. In one such embodiment, the inner catheter may be solid or hollow and may be constructed from any electrically insulating biocompatible material with one or more electrodes disposed on its outer surface. In another such embodiment, the inner catheter may be metal and may be coated with an electrical insulator except for at its distal tip or a region proximal to the distal tip. In embodiments where the inner catheter does not have a lumen or associated opening at its distal end, liquid medication, if any, would be provided only from the outer catheter. It can be appreciated that the full scope of use of such alternative embodiments would be evident to a person of skill in the art based upon the disclosure herein.

In some alternative embodiments, the hub 304 of the inner catheter may be removable from the proximal end of the inner catheter. In such embodiments, the removable hub includes electrical connectors that engage or make contact with electrical connectors on the inner catheter that connect with the one or more electrodes disposed on the outer surface of the catheter. It can be appreciated that any suitable engagement between the inner catheter and the removable hub is contemplated, including but not limited to, press-fitting, screw engagement, clamping with a further clamp, and others. The removable hub, when attached or engaged with the inner catheter, provides an injection port 306 into the lumen of the inner catheter. A removable inner catheter hub may have particular advantages, such as the ability for slidable connectors 309 or needles to be removed from the assembly while in use. In some embodiments, certain removable hubs may have differently configured electrical connectors or injection ports depending upon the intended application.

In an alternative embodiment where the inner catheter comprises a removable hub, a full needle having a fully defined outer circumference, such as a more typical straight needle, is used in place of the half-needle. In such an embodiment, the length of the inner catheter that passes through the outer catheter is fully circumferentially contained within the needle and does not communicate with the lumen of the outer catheter. With the hub of the needle engaged with the hub of the outer catheter, the distal end of the needle protrudes approximately 0.1-3 cm from distal end of the outer catheter, and the distal end of the inner catheter, in its first position, does not protrude beyond the distal tip of the needle. In use, once a muscle twitch has been elicited by electrically stimulating the tip of the needle, the electrical stimulation connection is removed from the needle and connected with the inner catheter to the stimulate the most distal electrode on the inner catheter. The inner catheter is then deployed to a second position beyond the distal tip of the needle until the muscle twitch is maintained. The needle may then be removed from the assembly by first removing the removable hub, followed by removing the slidable connector 309, and then pulling the needle through and out of the assembly. With the removable hub and slidable connector 309 removed, the needle does not encounter interference as it is removed from the proximal end of the assembly with the inner and outer catheters in-place. With the needle removed, the slidable connector may inserted over the inner catheter and into the hub of the outer catheter to secure the inner catheter position relative to the outer catheter. Prior to or after engaging the inserted slidable connector with the outer catheter, the removable hub of the inner catheter may be re-engaged. Once the removable hub of the inner catheter is re-engaged, it may be connected with a nerve stimulator to provide stimulation to the distal electrodes of the inner catheter in order to effect neuromodulation. It can be appreciated that the full scope of use of such alternative embodiments would be evident to a person of skill in the art based upon the disclosure herein.

In an alternative embodiment, a peel-away needle is used in place of the half-needle. A peel-away needle may be a needle that is fully circumferentially defined in a first configuration and, upon gradual or abrupt removal of a portion of the needle, a second configuration is no longer fully circumferentially defined. In some embodiments, a peel-away needle may split in half. In some embodiments, a peel-away needle may be used in place of the half-needle 212. In such embodiments, the assembly would be used as is described herein until the needle is removed. During the needle removal, the peel-away needle may gradually or abruptly split as the needle is pulled from the assembly. The splitting of the peel-away needle enables the needle to be removed without interfering with the slidable connector 309 or inner catheter hub 306. It can be appreciated that the full scope of use of such alternative embodiments would be evident to a person of skill in the art based upon the disclosure herein.

In an alternative embodiment, a needle fits inside of the inner catheter 300 and extends beyond both the outer catheter 200 and inner catheter 300 for insertion, after which the needle can be removed once the inner catheter 300 is placed in its second position. In such an embodiment, the needle would lock into position on the inner catheter hub 204, and the inner catheter would lock into position on the outer catheter via its slidable connector 309 engaging with the hub 206 of the outer catheter. In such an embodiment, the needle is a more typical straight needle and the half-needle is omitted. Each catheter would be configured and dimensioned to appropriately function according to this alternative embodiment and the disclosure herein. It can be appreciated that the full scope of use of such alternative embodiments would be evident to a person of skill in the art based upon the disclosure herein.

In an alternative embodiment, the outer catheter 200 further comprises an additional lumen attached or integrated with its outer perimeter such as is depicted in FIGS. 6 and 7A. The additional lumen may further provide an additional level of control over the localization of liquid medications, because medications could be delivered by the end of the additional lumen, by the end of the outer catheter 200, or by the end of the inner catheter 300. It can be appreciated that the full scope of use of such alternative embodiments would be evident to a person of skill in the art based upon the disclosure herein.

The catheter designs described herein each allow for delivering the neuromodulation and a liquid medication or medicinal nerve block either concurrently (simultaneously) or one treatment modality at a time. In some embodiments, either neuromodulation or medicinal nerve block are delivered. In some embodiments, both neuromodulation and medicinal nerve block are delivered. In some embodiments, the neuromodulation and medicinal nerve block are delivered concurrently (simultaneously). Systems delivering neuromodulation and nerve block concurrently may comprise a nerve stimulator and liquid medication delivery source, such as in infusion pump, operating in a synchronized manner such that the neuromodulation and medicinal nerve block are delivered simultaneously. In some embodiments, the neuromodulation and medicinal nerve block are delivered alternately, or one treatment modality at a time. Systems delivering neuromodulation and nerve block alternately may comprise a nerve stimulator and liquid medication delivery source, such as in infusion pump, operating in a desynchronized manner such that the neuromodulation and medicinal nerve block are delivered alternately.

Methods of neuromodulation and/or pain treatment may comprise the delivery of either neuromodulation or medicinal nerve block. Methods of neuromodulation and/or pain treatment may comprise the delivery of both neuromodulation and medicinal nerve block. In some embodiments, the neuromodulation and medicinal nerve block may be delivered simultaneously, or in a synchronized manner. In some embodiments, the neuromodulation and medicinal nerve block may be delivered alternately, or in a desynchronized manner. It can be appreciated that any timings or schedules for the delivery of neuromodulation and/or a liquid medication or nerve block are contemplated and may be decided upon by a practicing physician or person of skill in the art.

The muli-lumen or catheter-through-catheter design with multiple electrodes/lumens/openings allow these catheters to be placed/oriented alongside a nerve in a manner such that the active neuromodulating electrode used for delivering electrical pulse is closer to the central nervous system or spinal cord than the opening used for delivering medicine and thereby allowing uninterrupted electrical pulses to the spinal cord and concurrent injection of medicine further away (distal). In some embodiments, the neuromodulation proximal to the central nervous system or spinal cord is delivered concurrently with a liquid medication or nerve block at a position distal from the neuromodulation. In some embodiments, the neuromodulation proximal to the central nervous system or spinal cord is delivered alternatively from a liquid medication or nerve block at a position distal from the neuromodulation. In single-lumen embodiments, the treatment modality of the neuromodulation (nerve stimulation) and injection of a liquid medicine generally follow each other and are delivered in sequence over time.

The neuromodulation catheters may be used to treat any suitable pain, including, but not limited to, acute post-operative surgical pain, persistent pain after surgery, chronic pain, headaches, complex regional pain syndrome, post-traumatic pain, stump pain post-amputation, post-herpetic neuralgia, Peripheral field stimulation, electro-acupuncture, stimulation of the Dorsal Root Ganglion (DRG), stimulation of medial branch nerve, and Kilohertz stimulation.

While principles of the present disclosure are described herein with reference to illustrative embodiments for particular applications, it should be understood that the disclosure is not limited thereto. Those having ordinary skill in the art and access to the teachings provided herein will recognize additional modifications, applications, embodiments, and substitution of equivalents all fall within the scope of the embodiments described herein. Accordingly, the invention is not to be considered as limited by the foregoing description.

I claim:

1. A tubular catheter configured to provide neuromodulation, comprising:
an electrically non-conductive catheter wall circumscribing a lumen that extends from a proximal end of the tubular catheter to a distal end thereof;
a first electrically conductive portion formed on an exterior surface of the catheter wall adjacent the distal end;
a first electrical conductor extending from the proximal end to the distal end and electrically interconnected to the electrically conductive portion; and
one or more holes extending through the catheter wall located nearer the proximal end than the first electrically conductive portion.

2. The tubular catheter of claim 1 wherein a connector closes the proximal end, an injection port extending through the connector providing access to the lumen.

3. The tubular catheter of claim 2 wherein an electrically conductive contact post extends from an edge of the connector to the first electrical conductor.

4. The tubular catheter of claim 3 wherein the electrical conductor is a wire embedded with the catheter wall.

5. The tubular catheter of claim 1 wherein a second electrically conductive portion is formed on the exterior surface of the catheter wall at a location spaced from the first electrically conductive portion.

6. The tubular catheter of claim 5 wherein the first electrical conductor is electrically interconnected to the second electrically conductive portion.

7. The tubular catheter of claim 5 wherein a second electrical conductor is electrically interconnected to the second electrically conductive portion and the second electrical conductor is electrically isolated from the first electrically conductive portion.

8. A tubular catheter configured to provide neuromodulation, comprising:
a first electrically non-conductive catheter wall circumscribing a first lumen that extends from a proximal end of the first tubular catheter to a distal end thereof;
a first electrically conductive portion formed on an exterior surface of the first catheter wall adjacent the distal end;
a first electrical conductor extending from the proximal end to the distal end and electrically interconnected to the first electrically conductive portion;
a second electrically non-conductive catheter wall circumscribing a second lumen that extends from a proximal end of the second tubular catheter to a distal end thereof;
a second electrically conductive portion formed on an exterior surface of the second catheter wall adjacent the distal end; and
a second electrical conductor extending from the proximal end to the distal end and electrically interconnected to the second electrically conductive portion;
wherein the first catheter is affixed to the second catheter whereby the distal end of the second catheter is positioned between the distal end of the first catheter and the proximal end of the first catheter.

9. The tubular catheter of claim 8 further including a third electrically conductive portion formed on an exterior surface of either the first catheter wall or the second catheter wall.

10. The tubular catheter of claim 9 wherein the third electrically conductive portion is electrically isolated from both the first electrically conductive portion and the second electrically conductive portion.

11. The tubular catheter of claim 9 wherein the third electrically conductive portion is electrically interconnected to at least one of the first electrically conductive portion and the second electrically conductive portion.

12. The tubular catheter of claim 9 wherein the third electrically conductive portion is located between the first electrically conductive portion and the second electrically conductive portion.

13. The tubular catheter of claim 8 wherein the second catheter wall merges into the first catheter wall at an angle, α.

14. The tubular catheter of claim 13 wherein a is between about 10° and about 90°.

15. The tubular catheter of claim 13 wherein a portion of the first catheter wall and a portion of the second catheter wall is the same structure.

16. A pain mitigation system, comprising:
a catheter having:
an electrically non-conductive catheter wall circumscribing a lumen that extends from a proximal end of the tubular catheter to a distal end thereof;
a first electrically conductive portion formed on an exterior surface of the catheter wall adjacent the distal end;
a first electrical conductor extending from the proximal end to the distal end and electrically interconnected to the electrically conductive portion; and
one or more holes extending through the catheter wall located nearer the proximal end than the first electrically conductive portion;
a nerve stimulator configured to apply electrical pulses to the first electrically conductive portion; and
a port providing access to the lumen at the proximal end of the tubular catheter.

17. The pain mitigation system of claim 16 wherein the port is configured to receive a liquid analgesic.

18. The pain mitigation system of claim 17 wherein the nerve stimulator provides electric pulses having an amperage of between 0.1 and 20 milliamps and a pulse duration of between 0.1 and 1000 microseconds.

19. The pain mitigation system of claim 18 being configured to provide pain mitigate for a symptom selected from the group consisting of acute post-operative surgical pain, persistent pain after surgery, chronic pain, headaches, complex regional pain syndrome, post-traumatic pain, stump pain post-amputation (phantom limb pain), post-herpetic neuralgia, Peripheral field stimulation, electro-acupuncture, stimulation of the Dorsal Root Ganglion (DRG), stimulation of medial branch nerve, and Kilohertz stimulation or any condition that may benefit from neuromodulation or injection analgesic medication and combinations thereof.

20. A pain mitigation system, comprising:
a catheter having:
a first electrically non-conductive catheter wall circumscribing a first lumen that extends from a proximal end of the first tubular catheter to a distal end thereof;
a first electrically conductive portion formed on an exterior surface of the first catheter wall adjacent the distal end;
a first electrical conductor extending from the proximal end to the distal end and electrically interconnected to the first electrically conductive portion;
a second electrically non-conductive catheter wall circumscribing a second lumen that extends from a proximal end of the second tubular catheter to a distal end thereof;
a second electrically conductive portion formed on an exterior surface of the second catheter wall adjacent the distal end; and
a second electrical conductor extending from the proximal end to the distal end and electrically interconnected to the second electrically conductive portion;
wherein the first catheter is affixed to the second catheter whereby the distal end of the second catheter is positioned between the distal end of the first catheter and the proximal end of the first catheter.
a nerve stimulator configured to apply electrical pulses to the first electrically conductive portion; and
a port providing access to the one of the first lumen and the second lumen at the proximal end of the tubular catheter.

21. The pain mitigation system of claim 20 wherein the port is configured to receive a liquid analgesic.

22. The pain mitigation system of claim 21 wherein the nerve stimulator provides electric pulses having an amperage of between 0.1 and 20 milliamps and a pulse duration of between 0.1 and 1000 microseconds.

23. The pain mitigation system of claim 22 being configured to provide pain mitigate for a symptom selected from the group consisting of acute post-operative surgical pain, persistent pain after surgery, chronic pain, headaches, complex regional pain syndrome, post-traumatic pain, stump pain post-amputation (phantom limb pain), post-herpetic neuralgia, Peripheral field stimulation, electro-acupuncture, stimulation of the Dorsal Root Ganglion (DRG), stimulation of medial branch nerve, and Kilohertz stimulation or any condition that may benefit from neuromodulation or injection analgesic medication and combinations thereof.

24. The pain mitigation system of claim 21, wherein the liquid analgesic and nerve stimulation are provided simultaneously.

25. The pain mitigation system of claim 21, wherein the liquid analgesic and nerve stimulation are provided alternately.

26. An assembly for neuromodulation, comprising:
i) an outer catheter having an electrically non-conductive catheter wall circumscribing a lumen that extends through a proximal end of the outer catheter and through a distal end thereof, the distal end having an electrode disposed on an exterior surface of the catheter wall adjacent to the distal end; the proximal end having a hub with an injection port in connection with the lumen, and an electrical connector electrically connected to the electrode;
ii) a half-needle inserted through the lumen of the outer catheter, the half-needle having a distal tip extending beyond the distal end of the outer catheter, and a proximal hub engageable with the hub of the outer catheter, the hub of the half-needle having a second electrical connector;
iii) a deployable inner catheter partly circumferentially contained within the half-needle inserted through the lumen of the outer catheter;
the inner catheter having an electrically non-conductive catheter wall circumscribing an inner lumen that extends through a proximal end of the inner catheter and through distal end thereof, the distal end extending beyond the distal end of the outer catheter and having one or more electrodes disposed on an exterior surface of the catheter wall, where one of the electrodes is adjacent to the distal end;
the proximal end of the inner catheter extending through the proximal end of the outer catheter and having a hub with an injection port connected with the inner lumen, and electrical connectors independently electrically connected to the one or more electrodes; and
a slidable connector located in-between the proximal end of the outer catheter and the hub of the inner catheter which prevents movement of the inner catheter relative to the rest of the assembly when engaged.

27. The assembly of claim 26, wherein the injection port on the hub of the outer catheter extends longitudinally therefrom.

28. The assembly of claim 26 wherein the electrical connector of the outer catheter is connected electrically to the electrode of the outer catheter by a conductor embedded within the non-conductive wall of the outer catheter.

29. The assembly of claim 26, wherein the half-needle is electrically insulated along its length except at the tip.

30. The assembly of claim 29, wherein the second electrical connector is in electrical communication with the tip of the half-needle.

31. The assembly of claim 26 wherein the inner catheter comprises a first electrode disposed on an exterior surface of the catheter wall adjacent to the distal end and a second electrode proximal to the first electrode.

32. The assembly of claim 31, wherein the second electrode of the inner catheter is about 3 cm proximal to the first electrode of the inner catheter.

33. The assembly of claim 31 wherein a first electrical connector is electrically connected to the first electrode and a second electrical connector electrically connected to the second electrode.

34. The assembly of claim 33, wherein the first electrical connector is connected electrically to the first electrode of the inner catheter by a conductor embedded within the non-conductive wall of the inner catheter.

35. The assembly of claim 33, wherein the second electrical connector is connected electrically to the second electrode of the inner catheter by a conductor embedded within the non-conductive wall of the inner catheter.

36. The assembly of claim 26, wherein either of the injection ports on the outer and inner catheters are configured to accept a liquid medication injection.

37. The assembly of claim 26 wherein the slidable connector is engageable with the hub of the half-needle to prevent the inner catheter from moving relative to the half-needle and outer catheter when engaged.

38. The assembly of claim 26, wherein the half-needle is removable from the assembly when disengaged from the slidable connector of the inner catheter.

39. The assembly of claim 38, wherein the slidable connector of the inner catheter is engageable with the hub of the outer catheter with the half-needle removed to prevent the inner catheter from moving relative to the outer catheter when engaged.

40. A pain control system comprising:
i) the assembly of claim 26;
ii) one or more nerve stimulators.

41. The pain control system of claim 40 further comprising one or more liquid medication delivery sources connectable with the injection ports of the outer and inner catheters.

42. The pain control system of claim 41, wherein at least one of the liquid medication delivery sources is an infusion pump.

43. The pain control system of claim 42, wherein the infusion pump and nerve stimulators are synchronized to provide neuromodulation and liquid medication injection simultaneously.

44. The pain control system of claim 42, wherein the infusion pump and nerve stimulators are desynchronized to alternately provide neuromodulation and liquid medication injection.

45. The pain control system of claim 40 wherein one or more of the electrical connectors of the assembly are connected with the one or more nerve stimulators.

46. The pain control system of claim 45, wherein any connected electrical connectors are each independently supplied with an electrical signal of variable polarity, intensity, frequency, and pulse duration.

47. The pain control system of claim 46, wherein any connected electrodes can be independently stimulated with positive polarity, negative polarity, no polarity, or ground.

48. The pain control system of claim 46, wherein any connected electrodes can be independently stimulated at varying intensity from 0.01-30 mA, or from 0.01-20 mA, or from 0.1-10 mA or from 0.1-1 mA.

49. The pain control system of claim 46, wherein any connected electrodes can be independently stimulated at varying frequency (1 Hz-4999 Hz).

50. The pain control system of claim 46, wherein any connected electrodes can be independently stimulated at varying pulse durations ranging from 0.5 microseconds to 1000 microseconds.

51. A method of neuromodulation in a patient in need thereof using the pain control system of claim 40, comprising the following steps:
a. securing the inner catheter in a first position such that its proximal end extends beyond the proximal end of the outer catheter but not beyond the tip of the half-needle;
b. connecting the electrical connector of the half-needle to a nerve stimulator;
c. providing an electrical ground;
d. inserting the assembly into the patient toward a target nerve while stimulating the needle with electricity until the patient elicits a muscle twitch;
e. disconnecting the electrical nerve stimulator from the half-needle connector and connecting it to the connector associated with the most proximal electrode of the inner catheter to stimulate said electrode;
f. deploying the inner catheter from its first position to a second position more distal than the first position such that the muscle twitch is maintained;
g. removing the half-needle from the assembly; and
h. stimulating any combination of the electrodes by connection with the nerve stimulator to effect neuromodulation.

52. The method of claim 51 wherein the securing of step (a) is accomplished by engaging the slidable connector of the inner electrode with the hub of the half-needle.

53. The method of claim 51 wherein the electrical ground in step (c) is a surface skin electrode on the exterior of the patient.

54. The method of claim 51, wherein the electrical ground in step (c) is an electrode of the outer or inner catheter.

55. The method of claim 51, wherein the inserting in step (d) is performed under ultrasound or imaging guidance.

56. The method of claim 55, wherein the insertion trajectory in step (d) is toward the spinal cord or central nervous system.

57. The method of claim 55, wherein the insertion trajectory in step (d) is away from the spinal cord or central nervous system.

58. The method of claim 52, wherein the deploying step (f) comprises disengaging the slidable connector from the hub of the half-needle and sliding the inner catheter forward through the outer catheter and half-needle until the distal end of the inner catheter is approximately 3-8 cm beyond the tip of the half-needle.

59. The method of claim 58, wherein the removing step (g) comprises pulling the needle out of the assembly with the outer and inner catheters remaining in place, followed by engaging the slidable connector of the inner catheter with the hub of the outer catheter to prevent the inner catheter from moving relative to the outer catheter.

60. The method of claim 51, wherein the stimulating step (h) is performed by stimulating the electrode closest to the spinal cord or central nervous system.

61. The method of claim 60, wherein the electrical ground of the stimulating step (h) is a surface skin electrode on the exterior of the patient.

62. The method of claim 60, wherein the electrical ground of the stimulating step (h) is an electrode of the outer or inner catheter.

63. The method of claim 60, further comprising the step of injecting a liquid medication into one or both the injection ports of the outer and inner catheters.

64. The method of claim 63 wherein the liquid medication is injected during the neuromodulation step (h).

65. The method of claim 64, wherein the liquid medication is injected into the catheter having a distal end furthest from the neuromodulation.
